(19) United States
(12) Patent Application Publication
(10) Pub. No.: US 2024/0268646 A1
(43) Pub. Date: Aug. 15, 2024

(54) IMAGING ADAPTER FOR FLUORESCENCE IMAGING AND METHOD FOR MANUFACTURING AN IMAGING ADAPTER FOR FLUORESCENCE IMAGING

(71) Applicant: OLYMPUS Winter & Ibe GmbH, Hamburg (DE)

(72) Inventors: Matthias DISSEL, Hamburg (DE); Kulbir Singh RANDHAWA, Hamburg (DE)

(73) Assignee: OLYMPUS Winter & Ibe GmbH, Hamburg (DE)

(21) Appl. No.: 18/386,352

(22) Filed: Nov. 2, 2023

Related U.S. Application Data
(60) Provisional application No. 63/444,616, filed on Feb. 10, 2023.

(51) Int. Cl. A61B 1/04; A61B 1/06; A61B 1/07

(52) U.S. Cl. CPC A61B 1/043; A61B 1/0638; A61B 1/07

(57) ABSTRACT

An imaging adapter for fluorescence imaging including a guiding element body and a fiber bundle with multiple optical fiber. The imaging adapter connected with a camera head and configured to illuminate an operating area. The guiding element body includes a guiding slot extending through the guiding element body in the longitudinal direction. In a plane orthogonal to the longitudinal direction, the guiding slot is annular and segmented in a circumferential direction so that the guiding slot forms at least two separate guiding slot segments. In a proximal part of the fiber bundle, the optical fibers form a compact bundle, in a distal part of the fiber bundle the optical fibers are split apart from each other. The split apart optical fibers extend through the guiding slot segments to a distal end face of the guiding element body. Each guiding slot segment accommodates at least one of the optical fibers.

### CROSS-REFERENCE TO RELATED APPLICATION

The present application is based upon and claims the benefit of priority from U.S. Provisional Application No. 63/444,616 filed on Feb. 10, 2023, the entire contents of which is incorporated herein by reference.

### BACKGROUND

### Field

The present disclosure relates to an imaging adapter for fluorescence imaging and a method for manufacturing an imaging adapter for fluorescence imaging.

### Prior Art

In open surgery, video capturing units are often placed in the operating room to record the operating area. Such video capturing units usually comprise a camera head augmented by an optical imaging unit and an illumination unit.

A camera head usually comprises one or more optical sensors as well as a cone adapter connecting and securing the ocular funnels of optical imaging units to the camera head. Such optical imaging units may be devices for endoscopic procedures, such as rigid telescope type endoscopes having an optical assembly at their distal tip for forming an image of the specified field of view, and one or more relay lens units for relaying the image to the endoscope's ocular for view with the unaided eye, or alternatively, the camera head. For this purpose, the camera head comprises imaging optics that focus on the location of the virtual image projected by the ocular lens of the attached telescopes at their own focal distance. This focal distance is standardized between different types of telescopes, since they have to be usable with the unaided eye.

For the purpose of capturing the operating area, the optical imaging units are often designed to image the operating area with a predefined field of view at a predefined working distance. A typical example is the so-called exoscope, which resembles a short endoscope with an objective lens, a set of relay lens units and an eyepiece. The optical properties of the objective lens are quite different from those of objective lenses of endoscopes because of their very different focal lengths, since, unlike an endoscope, an exoscope is designed to operate outside a human body instead of inside the human body.

Since the camera head has its own imaging optics, an attachment lens system, also called a head lens, may be attached to the camera head's cone adapter that provides the combined optical system of the attachment lens system and the camera head's optical imaging system with the focal length and other optical properties necessary for viewing and recording the operating area.

In order to illuminate the operating area, the medical imaging systems usually comprise an illumination light generating unit comprising one or more light sources generating the illumination light. The illumination light is transported through a fiber bundle from the illumination light generating unit to the distal end of the illumination unit, where it exits the fiber bundle. Endoscopes and exoscopes usually do not comprise any light shaping units at the tip, so that the irradiance distribution at the field of operation is mainly defined by the irradiance distribution with which the light enters the fiber bundle from the light source.

Fluorescence imaging is a form of molecular imaging, which generally encompasses imaging methods for visualizing and/or tracking of molecules having specific properties that are used for molecular imaging. Such molecules can be substances that are endogenous to the body, or dyes or contrast agents that are injected into the patient. MRI and CT, for example, therefore also fall under the term “molecular imaging”. Fluorescence imaging as a variant of molecular imaging uses the property of certain molecules (fluorophores), which emit light of certain wavelengths when excited/absorbed by light of certain wavelengths.

Modern medical imaging systems such as the Olympus's Visera Elite 3 system implement the above-described versatility of endoscopic or open surgery imaging with various telescopes, exoscopes and imaging adapters for different applications that can be attached to the system's camera head, which is controlled by a central control unit (CCU). The telescopes, endoscopes and imaging adapters may have an illumination light connector to be connected to an illumination light generating unit of the medical imaging system via light fibers. The Visera Elite 3 system furthermore implements fluorescence imaging for fluorescence imaging guided surgery. The system's camera head includes sensors that are sensitive in the visible spectrum and in the near infrared spectrum, while the system's illumination light generating unit has light sources for white light as well as laser sources tuned for generating excitation light of typical dyes used in fluorescence imaging guided surgery, such as at 688 nm and 780 nm, which excite fluorescence dyes injected into patients before the procedure. After being excited, the dyes shed the excitation energy by emitting light at slightly longer wavelengths than the excitation light. Other wavelengths may be used as excitation wavelengths depending on the type of dye used. This can include wavelengths that are further inside the visible spectrum.

In order to illuminate the operating area, the optical fibers may be arranged around the imaging unit of the telescopes, exoscopes and imaging adapters in a ring like fashion. However, in such a ring shaped illumination unit, the axial alignment of the optical fibers cannot always be insured, leading to an inhomogeneous illumination distribution in the illuminated area. In addition, optical fibers often break when they are bend too much, making the arrangement of optical fibers around the imaging unit difficult.

### SUMMARY

An object is to provide an imaging adapter and a method to manufacture an imaging adapter, which illuminate the operating area homogeneously and with a high light intensity.

Such object can be solved by an imaging adapter for fluorescence imaging, comprising a guiding element body and a fiber bundle with multiple optical fibers, wherein the imaging adapter is configured to be connected with a camera head of a medical imaging system, wherein the imaging adapter is further configured to illuminate an operating area arranged in front of the imaging adapter in a longitudinal direction, the guiding element body comprises a guiding slot extending through the guiding element body in the longitudinal direction, in a plane orthogonal to the longitudinal direction the guiding slot is annular and segmented in a circumferential direction, so that the guiding slot forms at least two separate guiding slot segments, in a proximal part of the fiber bundle the optical fibers form a compact bundle, in a distal part of the fiber bundle the optical fibers are split apart from each other, the split apart optical fibers extend through the guiding slot segments to a distal end face of the guiding element body, and each guiding slot segment accommodates at least one of the optical fibers.

The imaging adapter can comprise an imaging unit and an illumination unit. The guiding element body and/or the fiber bundle can be part of the illumination unit. The guiding element body can be arranged concentrically around the imaging unit. In this way, the illumination unit can provide light for the operating area, while the imaging unit captures light from the operating area.

The guiding element body can ensure that the optical fibers are securely arranged in the imaging adapter and are guided towards the distal end face of the guiding element body, where the optical fibers emit the light to illuminate the operating area. By guiding the optical fibers in the guiding slot segments along the longitudinal direction, bends in the optical fibers can be prevented and it can be ensure that all optical fibers emit the light essentially in the same direction. In this way, the imaging adapter can achieve a homogeneous illumination. The guiding slot segments can simplify the arrangement of the optical fibers in the guiding slot and make it easier to homogenously distribute the optical fibers in the guiding slot. The optical fibers can be glued into the guiding slot segments. This can ensure a resilient construction of the imaging adapter.

The guiding element can be a solid of revolution. An axis of revolution of the solid of revolution can be parallel to the longitudinal direction. The axis of revolution can be an optical axis of the imaging adapter. The optical axis can run parallel to the longitudinal direction of the guiding element body. The guiding element body can comprise a cylindrical opening in its center, which can extend through the guiding element body in the longitudinal direction. The opening of the guiding element body can be configured to receive a tubular element, which can in turn receive the imaging unit or a component of the imaging unit.

The optical fibers can be guided in the guiding slot linearly and parallel to each other, and can be parallel to the optical axis of the imaging adapter, for a predetermined distance, wherein the predetermined distance can be at least 10 mm, such as at least 12 mm.

By guiding the optical fibers parallel to each other and parallel to the optical axis of the imaging adapter for a predetermined distance, the illumination can become more homogenous. In other words, slanted fiber ends at the distal end face can be prevented. In addition, by guiding the optical fibers parallel to each other for the predetermined distance, breakage of the optical fibers can be prevented. A minimal length of at least 10 mm, such as 12 mm, can be advantageous in order to reliably prevent the breakage of the optical fibers. The parts of the optical fibers guided parallel to each other can also be glued together. This can increase the optical fibers resistance to being broken.

According to an embodiment, the optical fibers can be guided in the guiding slot for at least part of the length along a conical surface, wherein a diameter of the conical surface can decrease towards the distal end face of the imaging adapter. In this way, the light can be focused towards the operating area.

In the plane orthogonal to the longitudinal direction, the guiding slot segments can have the shape of ring segments. The shape of ring segments can allow the optical fibers to be arranged essentially equidistant to the optical axis. The guiding slot segments can be symmetrical to each other. For example, the guiding slot segments can be axial symmetric around the optical axis or symmetric to a plane running through the optical axis.

The optical fibers in each guiding slot segment can form a fiber layer comprising multiple optical fibers. Thus, each guiding slot segment can comprise one fiber layer made up of a multitude of optical fibers, which can be glued together. For example, the fiber layers can have the shape of a sickle when viewed in the plane orthogonal to the longitudinal direction. The optical fibers in the fiber layer can be arranged abutting each other in the circumferential direction and the radial direction. The fiber layer can have a thickness of multiple optical fibers.

End faces of the optical fibers, which can be arranged at the distal end face of the guiding element body, can be grinded and polished. By grinding and polishing the end faces of the optical fibers, the illumination distribution can be further homogenized.

According to an embodiment, the guiding element body can comprise an outer ring element and an inner ring element that can form the guiding slot in between them in an assembled state. An inner radius of the outer ring element can be larger than the outer radius of the inner ring element. Thus, there remains a gap in between the outer ring and the inner ring which can form the guiding slot. The outer ring element and the inner ring element can be arranged concentrically to each other.

An outer surface of the inner ring element or an inner surface of the outer ring element can comprise at least two radial ledges that extend in a radial direction to an inner surface of the outer ring element or to an outer surface of the inner ring element, respectively. The radial ledges can serve as a positioning aid to arrange the inner ring element and the outer ring element concentrically to each other and in doing so can divide the guiding slot into the guiding slot segments. By providing two ledges, the annular guiding slot can be divided into the two ring shaped guiding slot segments.

According to an embodiment, the outer surface of the inner ring element or an inner surface of the outer ring element can comprise more than two radial ledges. By increasing the number of ledges, the number of ring shaped guiding slot segments can be increased as well. In this way, the illumination characteristics can be shaped as desired.

Only a first longitudinal part of the guiding slot can be segmented, wherein in a second longitudinal part the guiding slot can be continuous in the circumferential direction, wherein the first longitudinal part of the guiding slot can be arranged at a distal end of the imaging adapter. In other words, in a proximal part, the guiding slot can be continuous in the circumferential direction, while the guiding slot can be segmented into the guiding slot segments in a distal part of the guiding slot. This arrangement can make it easier to introduce the optical fibers into the guiding slot segments, as the proximal part offers more room for the optical fibers to be arranged, so that they can enter each guiding slot segment. In order to achieve this, the at least two radial ledges can extend only for a part of a longitudinal extension of the guiding slots.

According to an embodiment, the guiding element body can comprise an annular base component and a distal component, wherein the distal component can protrude from the base component in the longitudinal direction, wherein a distal end face of the distal component can be the distal end face of the guiding element body. The radial ledges can align the annular base component and the distal component concentrically to each other in the radial direction. The distal component and/or the annular base component can have a cylindrical shape. The annular base component can have a larger diameter than the distal component. The distal component can comprise the guiding slot. The annular base component can be configured to confine the optical fibers in the radial direction and guide them towards the guiding slot.

The imaging adapter further can comprise an imaging unit, an outer hull element and a tubular element, wherein the imaging unit can be arranged inside a central, e.g., cylindrical, opening of the tubular element, wherein the guiding element body can surround the tubular element concentrically and the outer hull element surrounds the guiding element body concentrically. This arrangement can provide a compact and functional way of guiding the fiber bundle and the optical fibers in the longitudinal direction and can simultaneously protect the optical fibers from being damaged. The tubular element can make it possible to arrange the imaging unit in the center of the imaging adapter, while the end faces of the optical fibers can be arranged concentrically around the imaging unit and can provide sufficient illumination.

The proximal part of the fiber bundle can enter the outer hull element through an opening in the outer hull element, wherein a point, where the optical fibers of the fiber bundle are split apart from each other, can be arranged inside the outer hull element. Thus, the fiber bundle can form a compact bundle outside the imaging adapter, which can prevent the fiber bundle from being damaged. The fiber bundle can be split apart only inside the imaging adapter, where the optical fibers cannot be damaged from outside sources.

According to an embodiment, the imaging adapter can comprise an ocular funnel, wherein the ocular funnel can be configured to be connected to an adapter of the camera head. The ocular funnel can provide an easy way of connection the imaging adapter with the camera head in order to quickly attach or remove the imaging adapter from the camera head and simultaneously provide the necessary optical quality for the imaging unit.

Such object can be further solved by a method for manufacturing an imaging adapter for fluorescence imaging, such as according to one of the previously mentioned embodiments, wherein the imaging adapter can be configured to be connected with or capable of being connected with a camera head of a medical imaging system, wherein the imaging adapter can be further configured to illuminate an operating area arranged in front of the imaging adapter in a longitudinal direction, wherein a guiding slot can be introduced into a guiding element body, wherein the guiding slot can extend through the guiding element body in the longitudinal direction, wherein in a plane orthogonal to the longitudinal direction, the guiding slot can be annular and segmented in a circumferential direction, so that the guiding slot can form at least two separate guiding slot segments, wherein a fiber bundle with multiple optical fibers can be prepared in a way that in a proximal part of the fiber bundle the optical fibers can form a compact bundle and in a distal part of the fiber bundle the optical fibers can be split apart from each other, wherein the split apart optical fibers can be placed inside the guiding slot segments so that the optical fibers can extend through the guiding slot segments to a distal end face of the guiding element body and each guiding slot segment can accommodate at least one of the optical fibers.

The same or similar advantages apply to the method of manufacturing an imaging adapter as previously mentioned with respect to the imaging adapter itself.

According to an embodiment, the guiding slot can be created by milling or laser ablation of material of the guiding element body. According to a different embodiment, the guiding slot can be created by fitting together an outer ring element and an inner ring element to form the guiding slot in between them. Both embodiments can provide an imaging adapter with a guiding element body, which can comprise a guiding slot with guiding slot segments.

### BRIEF DESCRIPTION OF THE DRAWINGS

Further characteristics will become apparent from the description of the embodiments together with the claims and the included drawings. Embodiments can fulfill individual characteristics or a combination of several characteristics.

The embodiments are described below, without restricting the general intent of the invention, based on exemplary embodiments, wherein reference is made expressly to the drawings with regard to the disclosure of all details that are not explained in greater detail in the text. The drawings show in:

FIG. 1 illustrates a schematic simplified representation of a camera head with a fluorescence imaging adapter,

FIG. 2 illustrates a schematic simplified perspective representation of an imaging adapter for fluorescence imaging,

FIG. 3 illustrates a schematic simplified perspective sectional drawing of the imaging adapter,

FIG. 4 illustrates a schematic simplified cross-sectional representation of the imaging adapter,

FIG. 5 illustrates a schematic simplified cross-sectional representation of the imaging adapter with optical fibers,

FIG. 6 illustrates a schematic simplified perspective representation of a guiding element body of an imaging adapter,

FIG. 7 illustrates a schematic simplified perspective cross-sectional representation of the guiding element body and

FIG. 8 illustrates a schematic simplified front view of the guiding element body with optical fibers.

In the drawings, the same or similar types of elements or respectively corresponding parts are provided with the same reference numbers in order to prevent the item from needing to be reintroduced.

### DETAILED DESCRIPTION

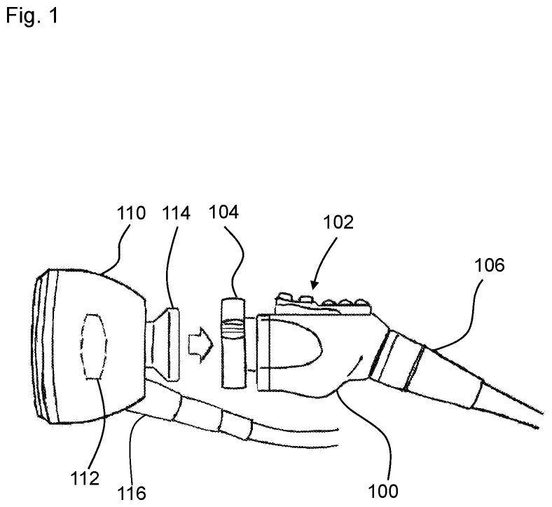

FIG. 1 illustrates a schematic representation of a medical imaging system with a medical imaging device, namely a combination of a camera head 100 and a fluorescence imaging adapter 110. The camera head 100 is configured for white light imaging as well as fluorescence imaging and is handheld or fixed in a mounting device. It and has control buttons 102 on the top of its housing, an adapter 104 for attachment of various optics systems at its front surface and a connecting cable 106 for power and signal transmission leading to a central control unit (not shown). In different embodiments, the buttons 102 may also be on the sides and/or the bottom of the housing. Since the camera head 100 is configured to receive telescope type endoscopes with eyepieces (ocular funnels), its adapter 104 may be configured to receive such eyepieces. The imaging optics of the camera head 100 is very shortsighted, since it is configured to have its focus at a location where the typically attached endoscopes project a virtual image to be viewed with the naked eye through the eyepiece. In context with the camera head 100, the location of such virtual images is typically inside the adapter 104, a short distance in front of the camera head's front face.

The fluorescence imaging adapter 110 differs from endoscopes and exoscopes in that it does not have imaging optics, i.e., it does not produce a virtual image. Instead, it provides a head lens or attachment lens in the form of a head lens system 112 having one or more individual lenses whose function it is to increase the focal length of the imaging optics of camera head 100, thus rendering the camera head 100 capable of viewing the operating field. Although the head lens system 112 itself does not provide a virtual image to be viewed with the naked eye, the fluorescence imaging adapter 110 has a standardized ocular funnel 114 on its rear side for the purpose of connecting to the adapter 104 of camera head 100.

Furthermore, the fluorescence imaging adapter 110 is equipped with a light guide cable 116 leading towards its front surface. The other end of the light guide cable 116 may be connected to an illumination light generating unit (not shown).

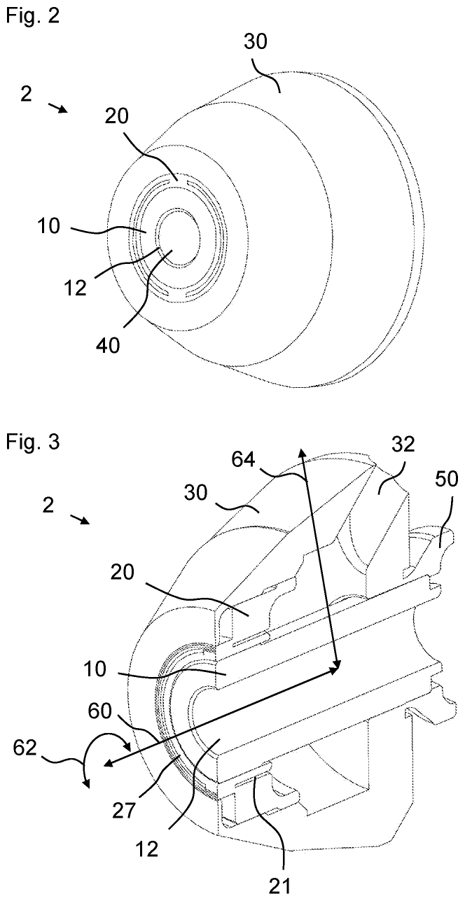

FIG. 2 shows a schematic simplified perspective view of another imaging adapter 2 for fluorescence imaging, which may be connected to the camera head 100. The imaging adapter 2 comprises a tubular element 10 with a central opening 12, which houses an imaging unit 40 or a component of the imaging unit 40. A guiding element body 20 is arranged concentrically around the tubular element 10 and a cone shaped outer hull element 30 is arranged concentrically around the guiding element body 20.

FIG. 3 shows a sectional drawing of the imaging adapter 2 of FIG. 2. In FIG. 3, it is visible that the opening 12 extends along a longitudinal direction 60 of the imaging adapter 2. The longitudinal direction 60 runs parallel to an optical axis of the imaging adapter 2. The tubular body 10, the guiding element body 20 and the outer hull element 30 are all essentially rotation bodies arranged around the central optical axis. The guiding element body 20 surrounds the tubular element 10 in a circumferential direction 62 of the imaging adapter 2 at a distal end face 27 of the imaging adapter 2. Similarly, the outer hull element 30 surrounds the guiding element body 20 in the circumferential direction 62 at the distal end face 27. Seen in a radial direction 64, the tubular element 10 forms an inner part, the guiding element body 20 a middle part and the outer hull element 30 an outer part of the imaging adapter 2. The imaging adapter 2 further comprises an ocular funnel 50 similar to the ocular funnel 114 in FIG. 1, which is configured to be connected to an adapter 104 of the camera head 100. The outer hull element 30 also features an opening 32, which is formed to receive a fiber bundle similar to the light guide cable 116 in FIG. 1. The guiding element body 20 comprises an annular guiding slot 21, which is formed to receive optical fibers of the fiber bundle, which will be explained in connecting with the following figures.

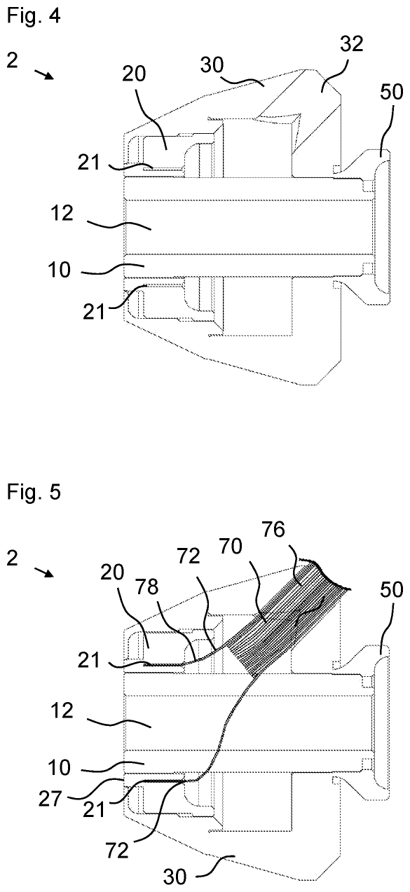

FIG. 4 shows a cross-sectional representation of the imaging adapter 2 of the FIGS. 2 and 3. Similarly, FIG. 5 shows the cross-sectional representation of FIG. 4 and additionally a fiber bundle 70, which enters the outer hull element 30 through the opening 32. As can be seen in FIGS. 4 and 5, the fiber bundle 70 takes the form of a compact bundle in a proximal part 76 of the fiber bundle 70. In this compact bundle, the individual optical fibers 72 form a single, compact string. However, in a distal part 78 of the fiber bundle 70, the optical fibers 72 are split apart from each other. In FIG. 5, only two of these split apart optical fibers 72 are shown to improve clarity of the drawing. However, the other optical fibers 72 of the fiber bundle 70 will be split apart as well and guided towards the guiding slot 21, where they are guided along the longitudinal direction 60. By guiding the optical fibers 72 along the longitudinal direction 60 for a certain distance, it is ensured that the light is emitted from the optical fibers 72 essentially in this longitudinal direction 60. Thus, a homogenous illumination of the operating area is provided.

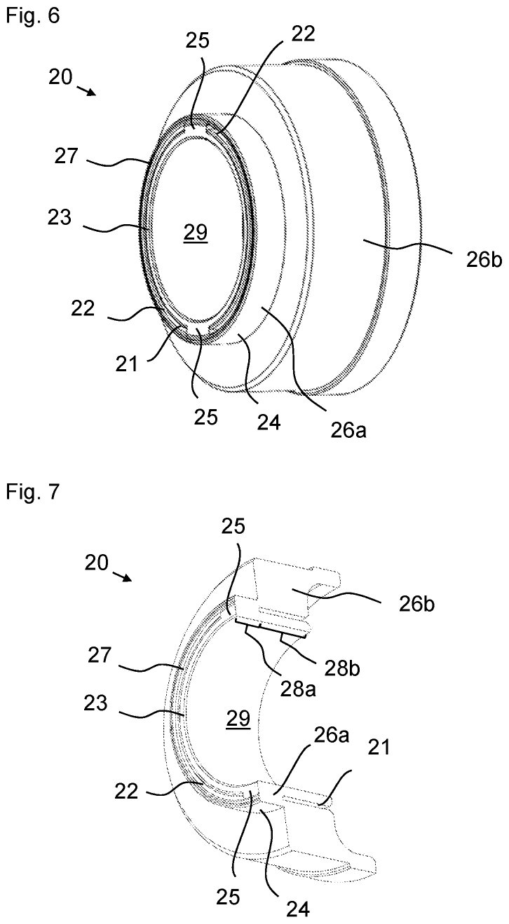

The arrangement of the optical fibers 72 in the guiding element body 20 as well as the features of the guiding element body 20 are further discussed with respect to the FIGS. 6, 7 and 8. As can be seen in the perspective representation in FIG. 6, the guiding element body 20 comprises an annular base component 26b and an annular distal component 26a protruding from the annular base component 26b. In the shown embodiment, the distal component 26a comprises an outer ring element 24 and an inner ring element 23. Inside the inner ring element 23, the guiding element body 20 comprises an opening 29 to receive the tubular element 10. Between the inner ring element 23 and the outer ring element 24 remains a gap, which forms the guiding slot 21 for the optical fibers 72. Two ledges 25 connect the inner ring element 23 and the outer ring element 24 in the radial direction 64. Doing so, the ledges 25 divide the guiding slot 21 into two separate guiding slot segments 22, which each have the shape of ring segments. In addition, the ledges 25 serve to arrange the inner ring element 23 and the outer ring element 24 in a concentric arrangement. In this guiding slot segments 22, the optical fibers 72, which are not shown in FIG. 6, are arranged.

FIG. 7 shows a sectional drawing of the guiding element body 20. As can be seen in this view, the ledges 25 between the inner ring element 23 and the outer ring element 24 extend only for a part of the length of the guiding element body 20. The ledges 25 are only present in a first longitudinal part 28a at the distal end face 27 of the imaging adapter 2. For a second longitudinal part 28b at a proximal end of the guiding element body 20, the ledges 25 are not present. Thus, in this second longitudinal part 28b, the guiding slot 21 forms a continuous annular slot, while in the first longitudinal part 28a the guiding slot 21 is divided into the two guiding slot segments 22. In different embodiments, there may be a greater number of ledges 25, which divide the guiding slot 21 into more than two guiding slot segments 22. In any case, each guiding slot segment 22 comprises at least one optical fiber 72. Most of the time, each guiding slot segment 22 will receive multiple or even dozens or hundreds of optical fibers 72.

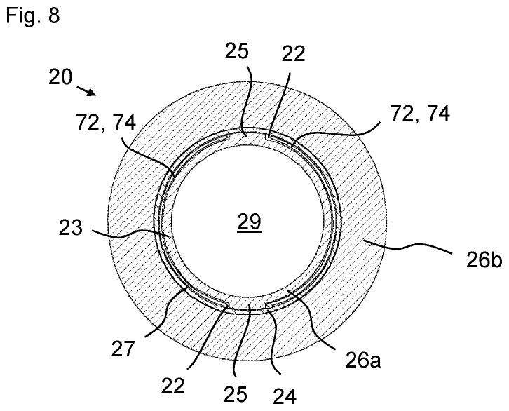

FIG. 8 shows a frontal view of the guiding element body 20. In this view, the ring segment shaped guiding slot segments 22 are easily visible. In FIG. 8, multiple optical fibers 72 are arranged in each guiding slot segment 22. The optical fibers 72 form a fiber layer 74 in each of the guiding slot segments 22. In the circumferential direction 62, this fiber layer 74 has the form of a ring segment or sickle. However, in the longitudinal direction 60, each optical fiber 27 of the fiber layers 74 is guided essentially parallel to each other so that the light is emitted from the end faces of the optical fibers 72 in essentially the same direction.

While there has been shown and described what is considered to be embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCES

2 imaging adapter
10 tubular element
12 central opening
20 guiding element body
21 guiding slot
22 guiding slot segment
23 inner ring element
24 outer ring element
25 ledge
26a distal component
26b base component
27 distal end face
28a first longitudinal part
28b second longitudinal part
29 opening
30 outer hull element
32 opening
40 imaging unit
50 ocular funnel
60 longitudinal direction
62 circumferential direction
64 radial direction
70 fiber bundle
72 optical fiber
74 fiber layer
76 proximal part
78 distal part
100 camera head
102 control buttons
104 adapter for attachment devices
106 connecting cable
110 fluorescence imaging adapter
112 head lens system
114 ocular funnel
116 light guide cable

What is claimed is:

1. An imaging adapter for fluorescence imaging, the imaging adapter comprising:
a guiding element body; and
a fiber bundle having a plurality of optical fibers;
wherein the imaging adapter is configured to be connected with a camera head of a medical imaging system;
the imaging adapter is further configured to illuminate an operating area arranged in front of the imaging adapter in a longitudinal direction;
the guiding element body comprises a guiding slot extending through the guiding element body in the longitudinal direction;
in a plane orthogonal to the longitudinal direction the guiding slot is annular and segmented in a circumferential direction so that the guiding slot forms at least two separate guiding slot segments;
in a proximal part of the fiber bundle the optical fibers form a compact bundle;
in a distal part of the fiber bundle the optical fibers are split apart from each other;
the split apart optical fibers extend through the guiding slot segments to a distal end face of the guiding element body; and
each guiding slot segment accommodates at least one of the optical fibers.

2. The imaging adapter according to claim 1, wherein the plurality of optical fibers are guided in the guiding slot linearly and parallel to each other.

3. The imaging adapter according to claim 2, wherein the plurality of optical fibers are guided in the guiding slot parallel to an optical axis of the imaging adapter for a predetermined distance.

4. The imaging adapter according to claim 3, wherein the predetermined distance is 3 mm to 10 mm.

5. The imaging adapter according to claim 4, wherein the predetermined distance is 4 mm to 6 mm.

6. The imaging adapter according to claim 1, wherein, in the plane orthogonal to the longitudinal direction, the guiding slot segments have a shape of ring segments.

7. The imaging adapter according to claim 1, wherein the plurality of optical fibers in each guiding slot segment form a fiber layer.

8. The imaging adapter according to claim 1, wherein end faces of the optical fibers, which are arranged at the distal end face of the guiding element body, are grinded and polished.

9. The imaging adapter according to claim 1, wherein the guiding element body comprises an outer ring element and an inner ring element that together form the guiding slot.

10. The imaging adapter according to claim 9, wherein an outer surface of the inner ring element or an inner surface of the outer ring element comprises at least two radial ledges that extend in a radial direction to an inner surface of the outer ring element or to an outer surface of the inner ring element, respectively.

11. The imaging adapter according to claim 1, wherein only a first longitudinal part of the guiding slot is segmented, and in a second longitudinal part the guiding slot is continuous in the circumferential direction.

12. The imaging adapter according to claim 11, wherein the first longitudinal part of the guiding slot is arranged at a distal end of the imaging adapter.

13. The imaging adapter according to claim 1, wherein the guiding element body comprises an annular base component and a distal component, the distal component protrudes from the base component in the longitudinal direction, and a distal end face of the distal component is the distal end face of the guiding element body.

14. The imaging adapter according to claim 1, further comprising:
an imaging unit,
an outer hull element, and
a tubular element,
wherein the imaging unit is arranged inside a central, opening of the tubular element, and
the guiding element body surrounds the tubular element concentrically and the outer hull element surrounds the guiding element body concentrically.

15. The imaging adapter according to claim 14, wherein the proximal part of the fiber bundle enters the outer hull element through an opening in the outer hull element, and a point, where the optical fibers of the fiber bundle are split apart from each other, is arranged inside the outer hull element.

16. The imaging adapter according to claim 1, further comprising an ocular funnel configured to be connected to an adapter of the camera head.

17. A method for manufacturing an imaging adapter for fluorescence imaging, the method comprising:
inserting an optical fiber bundle into a guiding element body of the imaging adapter;
splitting a distal end portion of the optical fiber bundle into a plurality of individual optical fibers; and
inserting at least some of the plurality of individual optical fibers through each of a plurality of annular guiding slot segments formed at a distal end face of the guiding element body of the imaging adaptor.

18. The method according to claim 17, wherein the guiding slot segments are created by milling or laser ablation of material of the guiding element body.

19. The method according to claim 17, wherein the guiding slot segments are created by fitting together an outer ring element and an inner ring element to form the guiding slot.
